(19) United States
(12) Patent Application Publication
(10) Pub. No.: US 2019/0136299 A1
(43) Pub. Date: May 9, 2019

(54) METAGENOMIC METHOD FOR IN VITRO DIAGNOSIS OF GUT DYSBIOSIS

(71) Applicant: OSPEDALE PEDIATRICO BAMBINO GESU', Roma (IT)

(72) Inventors: Lorenza PUTIGNANI, Roma (IT); Federica DEL CHIERICO, Roma (IT)

(21) Appl. No.: 16/310,760

(22) PCT Filed: Jun. 16, 2017
(86) PCT No.: PCT/IT2017/000119

§ 371 Date: Dec. 17, 2018

(30) Foreign Application Priority Data
Jun. 16, 2016 (IT) 102016000062306

(51) Int. Cl. C12Q 1/689

(52) U.S. Cl. CPC C12Q 1/689

(57) ABSTRACT

The present invention concerns a metagenomic method for in vitro diagnosis of gut dysbiosis able to assign a dysbiosis degree in comparison to healthy subjects.

The present invention concerns a metagenomic method for in vitro diagnosis of gut dysbiosis. Particularly, the present invention concerns a metagenomic method for in vitro diagnosis of gut dysbiosis able to assign a dysbiosis degree in comparison to healthy subjects.

Gut microbiota is a complex community of microorganisms that live in the human gut. Gut microbiota is generally comparable for individuals in selected groups of population, with recent evidence supporting gut microbiota health associated to a state of eubiosis or dysbiosis, depending on physiological or disease-related conditions, respectively.

Notably, deviations from eubiosis can result in a transient or permanent microbiota imbalance known as dysbiosis, which has been linked to several disorders, including inflammatory bowel disease (IBD), such as Crohn's disease (CD), ulcerative colitis (UC), or irritable bowel syndrome (IBS), obesity, nonalcoholic steatohepatitis, type I and type II diabetes, cystic fibrosis, autoimmune diseases or neurological disorders. Traditionally, evaluation of gut microbiota composition has been based on culture-based techniques and more recently on culture-independent techniques such as high-throughput next-generation sequencing (NGS).

The use of these methods has significantly improved the understanding of the role of gut microbiota in health and disease, especially during pediatric age; for example, small intestinal bacterial overgrowth and altered intestinal microbiota are implicated in subgroups of patients with functional bowel disorders.

Microbial profiling under the host—microbe and microbe—microbe interplays is now one of the most promising laboratory tool to describe symbiosis-dysbiosis shift of gut microbiota (Putignani et al., 2016). Gut microbiota has several metabolic, protective, structural, and mucosal functions. When symbiosis switches to gut dysbiosis, the imbalance involves the liver, adipose tissue, and the immune system (IS), and the gut ecosystem loses many bacterial species altering homeostasis.

Hence, after perturbations, the gut microbiota ecosystem can shift to a state of dysbiosis, in which commensal protective function, structural and histological role, and metabolic activities manifest impaired concerted mechanisms. This can involve overgrowth (blooming) of otherwise under-represented or potentially harmful bacteria (i.e., pathobionts), induced by intrusion or disappearance of individual members (i.e., invading bacterial strains during maturation of infant gut microbiota); shifts in relative bacterial abundances by external stimuli; and mutation or horizontal gene transfer can affect healthy status of the subjects. These alterations influence significantly the overall functionality of microbiota, by enhancing the fitness of certain pathogens or commensal stabilizers.

Some methods for detecting gut microbial composition have been described up to now. In the past, the analysis of bacterial ecosystems was based on the microbial growth on laboratory culture media, but the great limitations of this technique resides in the inability to culture the 80% of stool bacteria (Sekirov et al., 2006) As a consequence, new molecular techniques have been developed. In terms of qualitative measurements of the microbiota, methods such as fingerprinting (denaturing gradient gel electrophoresis), terminal restriction fragment length polymorphism, ribosomal intergenic spacer analysis, and 16S ribosomal RNA sequencing are widely used (Blaut et al., 2002). The new automated massive technologies, based on the 16S ribosomal RNA gene sequencing, present in all prokaryotes, can offer a cost-effective solution for rapid sequencing and identification of all bacterial species of the gut. Metagenomics relates to culture-independent studies of microbial communities to explore microbial consortia that inhabit specific niches in plants or in animal hosts, such as mucosal surfaces and human skin.

For quantitative measurements of gut microbiota bacteria distribution, techniques such as fluorescence in situ hybridization, catalyzed reporter deposition-fluorescence in situ hybridization, quantitative polymerase chain reaction, and scanning electron microscopy in situ hybridization have been used (Peter and Sommaruga, 2008). These methods are able to detect change in total number of microorganisms, change in gut microbiota species, or allow to address the presence or absence of specific bacterial species. However, the estimation of these differences need to be established compared to reference individuals selected amongst healthy subjects.

In recent years, the knowledge regarding species and functional composition of the human intestinal microbiome has increased rapidly, but very little is still known about the composition of microbiome in term of level of normobiosis conditions and inter-individual variability associated to geographical and diet-dependent conditions.

Arumugam and colleagues (Arumugam et al., 2011) characterized variations in the composition of the intestinal microbiota in 39 individuals from four continents by analyzing the fecal metagenome. The authors proposed that the intestinal microbial community could be stratified into three groups, called enterotypes. Each of these three enterotypes is identifiable by the variation in the levels of one of three genera: Bacteroides (enterotype 1), Prevotella (enterotype 2), and Ruminococcus (enterotype 3). Despite the stability of these three major groups, their relative proportions and the species present are highly variable between individuals. Therefore, Siezen and Kleerebezem proposed a new term called “faecotypes” instead of “enterotypes,” since it is known that the microbial abundance and composition changes dramatically throughout the gut intestinal tract, and perhaps “enterotypes” may not reflect the microbial composition of the whole intestine (Siezen and Kleerebezem, 2011). Although the intestinal microbiota is stable in adulthood, it undergoes fluctuations during childhood and old age. In children, the type of bacteria colonizing the intestine is defined very early according to the type of delivery and feeding modality (Del Chierico et al., 2015).

It is also known that in elderly individuals, there is a decreasing quantity and diversity of species of Bacteroides and Bifidobacterium and an increase in facultative anaerobe bacteria. Increase of these bacteria genus is harmful to host since they present high proteolytic activity, which is responsible for putrefaction of large bowel (Woodmansey, 2007). However, at present, there are no studies on gut microbiota which are able to provide a reference microbiota reservoir for the proper description of intestinal eubiosis profiles, to be compared, as reference, to the profiles of patients with disorders and gastrointestinal diseases, in order to detect gut dysbiosis and/or the grade of dysbiosis in term, for example, of mild, moderate and severe dysbiosis. Gut dysbiosis refers to a microbial imbalance inside the intestine in comparison to healthy gut microbiota profiles.

In the light of the above it is therefore apparent the need to provide for new methods for the diagnosis of gut dysbiosis able to overcome the disadvantages of known methods.

According to the present invention, the gut microbiota profiling of healthy subjects has been detected by metagenomics. Particularly, gut microbiota composition (or profiling) has been detected both qualitatively and quantitatively for every taxonomic level, i.e. phylum, family and species. It has been found that gut microbiota composition is independent on gender, however it is dependent on age of the subjects whom the microbiota belongs for all taxonomic levels, i.e. phylum, family and species taxonomic levels.

In addition, it has been found that gut microbiota composition of a healthy subject does not change over time at all taxonomic levels.

On the basis of the above, the present invention provides the essential criteria for setting up a methagenomic method which is surprisingly able to detect every grade of gut dysbiosis of a patient in a significantly statistical way in comparison to a healthy control group. Specifically, gut microbiota composition (or profiling) of a patient can be compared with gut microbiota composition of healthy subjects who are the same or similar age as the patient. A statistically significant difference between gut microbiota of a patient and gut microbiota of healthy subjects is detected at family and species taxonomic levels for every grade of dysbiosis, whereas a statistically significant difference is obtained only in patients with very serious dysbiosis at phylum taxonomic level.

The healthy subjects should be selected preferably among those having overlapping dietary habits (the same or similar), since gut microbiota can be influenced by nutrition patterns and environmental stimuli. For instance, dietary habits depend on geographical area and culture which results in different kinds of diet such as, for example, Mediterranean diet, Japonese diet, Western diet, African diet. Therefore, the healthy subjects should be selected among those having the same kind of diet, with an income of nutrients pretty balanced, resembling a complete omnivore diet, rather than prevalently vegetarian or even vegan.

Preferably, the healthy subjects could be selected among those coming and living in the same geographical area, for instance in the same country or nation, in addition to being selected on the basis of the dietary habits, possibly excluding groups of individuals characterized by highly strict dietary habits.

It is therefore a specific object of the present invention a method for providing a gut microbiota reference control tool of healthy subjects for in vitro diagnosis of gut dysbiosis index or percentage, said method comprising or consisting of:

a) clustering gut biological samples of healthy subjects in one or more clusters wherein, when the age of the healthy subjects is less than 17 or 17±2 years, preferably from about 18 months to less than 17 or 17±2 years, the gut biological samples belong to healthy subjects having an age difference less than 4 years, preferably less than 3 years, more preferably less than 2 years, among them in each cluster, and/or in a further cluster wherein the gut biological samples belong to healthy subjects whose age ranges from 17, or 17±2 years, to 70 or 70±2 years;

b) detecting by metagenomics the identity and frequency of all phyla, families and species of gut microbiota in the gut biological samples of each of said healthy subjects of each of said one or more clusters; and

c) calculating the median values of the operational taxonomic units distribution for each of said one or more clusters and/or said further cluster.

The cluster according to the invention is therefore an homogeneous cluster, i.e. when identified by the Wald's method, it is characterized by multivariate data revealing characteristics of any structure or patterns present (e.g. microbiota profiles generating subgroups belonging to the same clustering tree node) (Agresti, A. 2007. An Introduction to Categorical Data Analysis, 2nd ed., New York: John Wiley & Sons. Everitt, B. 2011. Cluster analysis. Chichester, West Sussex, U.K: Wiley. ISBN 9780470749913).

Each cluster can comprise biological samples of at least 10 subjects.

For each cluster, a median value of the operational taxonomic units distribution is obtain for each of said all phyla, families and species, i.e. three median values of the operational taxonomic units distribution are obtained for each cluster.

All phyla, families or species are all phyla, families and species detectable on the basis of the knowledge at the time of detection.

According to an embodiment of the present invention, said one or more clusters can be clusters wherein the gut biological samples belong to healthy subjects whose age ranges from 2 years to less than 4 years, from 4 years to less than 7 years, from 7 years to less than 9 years, from 9 years to less than 11 years, from 11 years to less than 13 years, from 13 years to less than 17 years, and/or from 17 years to 70 years.

Therefore, the method of the present invention can be used for in vitro diagnosis of gut dysbiosis index or percentage in pediatric age or childhood as well as in adulthood.

Gut biological samples to be used in the method of the present invention can be faecal samples, gut tissue samples, preferably faecal samples.

According to the present invention, the healthy subjects preferably come from the same Nation.

The present invention concerns also a gut microbiota reference control tool of healthy subjects for in vitro diagnosis of gut dysbiosis index or percentage, said reference control tool comprising or consisting of the median values of the operational taxonomic units distribution of all phyla, families and species, which are detected by metagenomics, of gut microbiota in gut biological samples of healthy subjects, wherein said gut biological samples are clustered in one or more clusters wherein, when the age of the healthy subjects is less than 17 or 17±2 years, preferably from about 18 months to less than 17 or 17±2 years, the gut biological samples belong to healthy subjects having an age difference less than 4 years, preferably less than 3 years, more preferably less than 2 years, among them in each cluster, and/or in a further cluster wherein the gut biological samples belong to healthy subjects whose age ranges from 17, or 17±2 years, to 70 or 70±2 years; wherein said median values of the operational taxonomic units distribution are the median values of the operational taxonomic units distribution for each of said one or more clusters and/or said further cluster.

According to an embodiment of the present invention, in the gut microbiota reference control tool, said one or more clusters can be clusters wherein the gut biological samples belong to healthy subjects whose age ranges from 2 years to less than 4 years, from 4 years to less than 7 years, from 7 years to less than 9 years, from 9 years to less than 11 years, from 11 years to less than 13 years, from 13 years to less than 17 years, and/or from 17 years to 70 years.

As mentioned above, gut biological samples to be used according to the present invention are faecal samples, gut tissue samples, preferably faecal samples.

According to the present invention, the healthy subjects preferably come from the same Nation.

The present invention concerns also a method for in vitro diagnosis of gut dysbiosis index or percentage comprising or consisting of:

a) detecting by metagenomics the identity and frequency of all detectable phyla, families and species of gut microbiota in more than two, preferably three, gut biological samples of a patient which are collected in consecutive days;

b) calculating the median values of operational taxonomic units distribution of said all detectable phyla, families and species of said gut biological samples of the patient;

c) calculating the dissimilarity index or percentage of the median values of the operational taxonomic units distributions of gut microbiota of the patient in comparison with the median values of the operational taxonomic units distribution of a cluster of the gut microbiota reference control tool of healthy subjects as defined in anyone of the claims 5-8, wherein said cluster is that in which the age of the patient falls in the age range of the healthy subjects of the same cluster.

The dissimilarity index or percentage is calculated by comparing data which refer to the same taxonomic level, i.e. phylum, family or species and then to all phyla, families and species of gut microbiota of the patient compared to controls.

In detail, the dissimilarity index or percentage can be calculated for said all phyla, families and species of gut microbiota of the patient by the formula:

Z=(½×Σ(f_{case}−f_{controls})²)^{1/2}

or

Z=(½×Σ(f_{case}−f_{controls})²)^{1/2}×100

wherein f_{case} is the median value of the operational taxonomic units distribution of said all phyla, families and species of gut microbiota of the patient;

and f_{controls} is the median value of the operational taxonomic units distribution of all phyla, families and species of gut microbiota of the cluster of the gut microbiota reference control tool of healthy subjects as defined above, wherein said cluster is that in which the age of the patient falls in the age range of the healthy subjects of the same cluster.

According to the method of the present invention, the patient preferably comes from the same Nation of the healthy subjects of the control tool as defined above.

The index or percentage varies from 0 to 100 or from 0 to 1: the value 0 means no dissimilarity and the value 100 or 1 means max dissimilarity.

The methods for detecting gut microbiota prevalently qualitatively are well known. For example, fingerprinting (denaturing gradient gel electrophoresis), terminal restriction fragment length polymorphism, ribosomal intergenic spacer analysis, and 16S ribosomal RNA sequencing (Blaut et al., 2002) are known.

Particularly, gut microbiota can be detected by amplifying and pyrosequencing V1-V3 region of 16S ribosomal RNA gene of the microorganisms contained in a gut biological sample according to Ercolini et al, 2012. In a typical gut metagenomic experiment, after DNA extraction from fecal sample, a short segment of the 16S rRNA is amplified. By amplifying and sequencing selected regions within 16S rRNA genes, bacteria can be identified. The identity at phylum, family and species taxonomic level and frequency of bacteria in a sample are determined by assigning reads to known 16S rRNA database sequences via sequence homology. After homology process, however, frequencies of reads and, hence, frequencies of bacteria are assigned by using Quantitative Insights into Microbial Ecology (QIIME 1.8.0, as below reported in detail. Therefore, the method according to the present invention can be a metagenomic method.

The present invention now will be described by an illustrative, but not limitative way, according to preferred embodiments thereof, with particular reference to enclosed drawings, wherein:

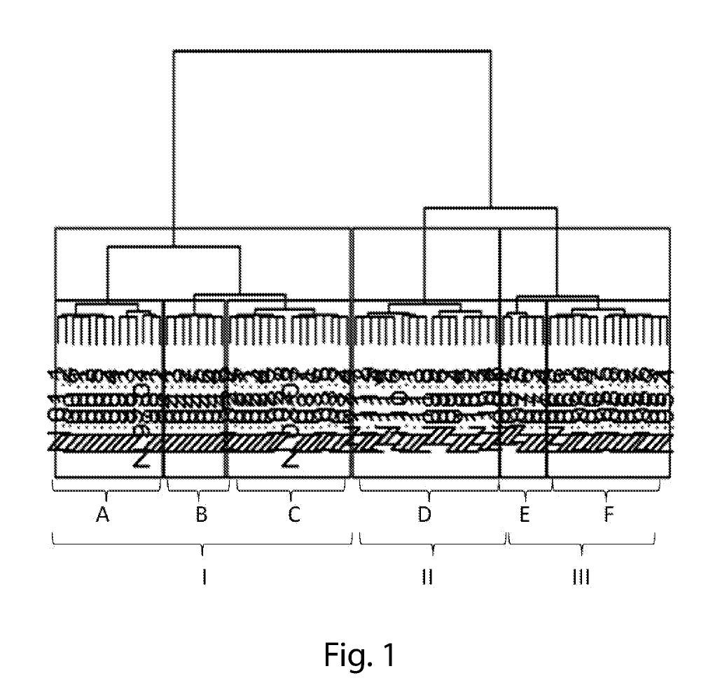

FIG. 1.—Clustering of controls by Wald's method at L2 taxon level—3 groups (curly brackets from I to III) 6 groups (curly brackets from A to F).

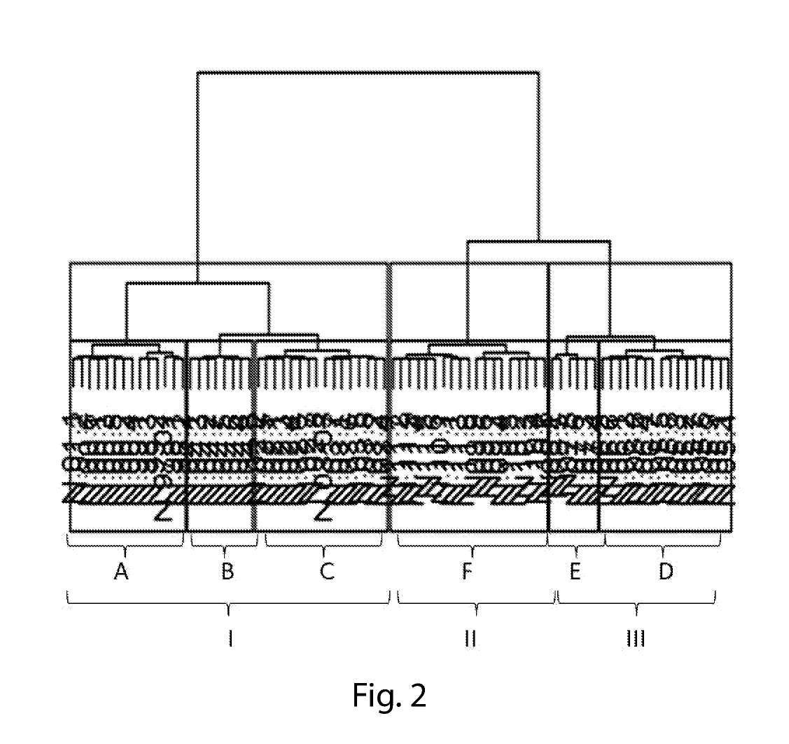

FIG. 2.—Clustering of controls by Wald's method at L5 taxon level—3 groups (curly brackets from I to III) 6 groups (curly brackets from A to F).

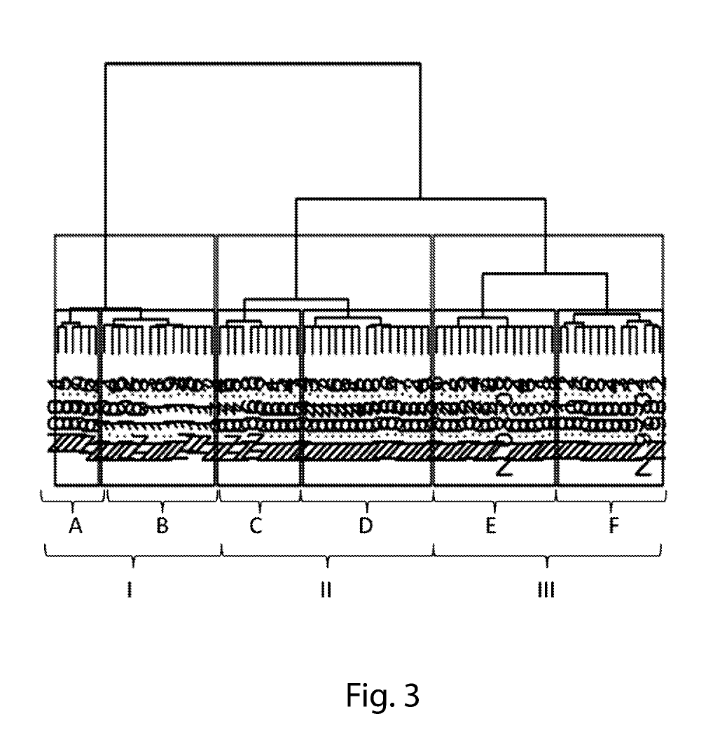

FIG. 3—Clustering of controls by Wald's method at L6 taxon level—3 groups (curly brackets from I to III) 6 groups (curly brackets from A to F).

### EXAMPLE 1: STUDY OF MICROBIOTA PROFILING

Introductory Materials and Methods for Microbiota Profiling Generation.

1. Relative Abundances of OTUs Calculated by Metagenomics.

Three and one stool sample was collected and processed from each patient and each reference subject, respectively. Genomic DNA was isolated from the entire set of 96 samples, using the QIAamp DNA Stool Mini Kit (Qiagen, Germany). The V1-V3 region of 16S ribosomal RNA (rRNA) locus was amplified for next pyrosequencing step on a 454-Junior Genome Sequencer (Roche 454 Life Sciences, Branford, USA). Reads were analyzed by Quantitative Insights into Microbial Ecology (QIIME, v.1.8.0), grouped into operational taxonomic units (OTUs) at a sequence similarity level of 97% by PyNAST for taxonomic assignment, and aligned by UCLUST for OTUs matching against Greengenes database (v. 13.8).

Genomic DNA Extraction.

Genomic DNA was extracted from all faecal samples. Stools were resuspended into 1.5 ml PBS, homogenized by vortexing for 2 min and centrifuged at 20,800×g. After supernatant removal, pellet was resuspended into 500 μl of PBS added by 500 μl of Beads/PBS (1 mg/μl, w/v) (Glass Beads, acid-washed SigmaAldrich). The 1:1 mixture was homogenized by vortexing for 2 min and centrifuged at 5200×g for 1 min. The supernatant was collected, and treated for one freeze-thaw cycle (−20° C./70° C.) for 20 min each step. After centrifugation at 5200×g for 5 min, the supernatant was subjected to QIAamp DNA Stool Mini Kit (Qiagen, Germany) extraction, according to manufacturer's instructions. DNA was eluted into 50 μl purified H₂O (Genedia, Italy) and its yield quantified using a NanoDrop ND-1000 spectrophotometer (NanoDrop Technologies, Wilmington, Del.). DNA was adjusted to 10 ng/μl concentration and used as template for successful 16S Metagenomic 454 Sequencing Analyses.

Amplicon Library Preparation and Pyrosequencing.

Gut microbiome was investigated by pyrosequencing V1-V3 regions of 16S rRNA gene (amplicon size 520 bp), on a GS Junior platform (454 Life Sciences, Roche Diagnostics, Italy), according to the pipeline described elsewhere (Ercolini et al, 2012). In a typical gut metagenomic experiment, after DNA extraction from fecal sample, a short segment of the 16S rRNA is amplified. By amplifying and sequencing selected regions within 16S rRNA genes, bacteria can be identified. The identity at phylum, family and species taxonomic level and frequency of bacteria in a sample are determined by assigning reads to known 16S rRNA database sequences via sequence homology.

For the metagenomics analysis needs:

QIAAMP DNA STOOL MINI KIT (Qiagen) for DNA extraction from fecal samples; Fast Start Hi-Fi PCR system dNTP Pack (Roche diagnostics) for 16S rRNA amplification;

EmPCR Kit Oil and Breaking Kit, EmPCR Kit EmPCR Reagents (Lib-L), EmPCR Bead Recovery Reagents, Sequencing Kit Reagents and Enzymes, Sequencing Kit Packing Beads and Supplement CB, Sequencing Kit Buffers, PicoTiterPlate Kit (Roche diagnostics) for pyrosequencing reactions.

Bioinformatics.

A first result filtering was performed using the 454 Amplicon signal processing; sequences were then analyzed by using Quantitative Insights into Microbial Ecology (QIIME 1.8.0) software (Caporaso et al., 2010). In order to guarantee a higher level of accuracy in terms of Operational Taxonomic Units (OTUs) detection, after demultiplexing, reads with an average quality score lower than 25, shorter than 300 bp, and with an ambiguous base calling were excluded from the analysis.

Sequences that passed the quality filter were denoised (Reeder et al., 2010) and singletons were excluded. The OTUs defined by a 97% of similarity were picked using the uclust method (Edgar et al., 2010) and the representative sequences were submitted to PyNAST, for the sequence alignment the used method was UCLUST and the database for OTUs matching was greengenes (v 13.8). The last step consisted in building an OTU table with the absolute abundance of each OTU across all samples, followed by the taxonomic assignment: 6 levels of deep taxonomy (from kingdom to species), unassigned OTUs and unspecified levels were considered.

Ecological diversity for each sample was assessed by: i) number of OTUs obtained from each samples; ii) Shannon index, giving the entropy information of the observed OUT abundances and account for both richness and eveness; Chao1 metric estimating species richness; iv) phylogenetic distance (PD_whole_tree) to assess quantitative measure of phylogenetic diversity; v) observed species metric, counting unique OTUs found in the sample; vi) Good's coverage, measuring the percentage of the total species represented in a sample. The β-diversity, representing the comparison of microbial communities based on their dissimilar composition, was calculated by unweighted and weighted UNIFRAC and Bray-Curtis algorithms. The α and β diversity and the Kruskal Wallis test were performed by QIIME software, using “alpha_rarefaction.py, beta_diversity_through_py, group_significance.py” scripts. Furthermore, to measure the robustness of the results a jackknifing analysis was performed. To measure the robustness of this data a jackknifing analysis was performed on data subsets, and the resulting Unweighted Pair Group Method with Arithmetic (UPGMA) tree was compared with the entire data set tree (jackknifed_beta_diversity.py—i otus/otu_table.txt—t otus/rep_set.tre—m Fasting_Map.txt−o wfjack—e). This process was repeated with many random subsets of data (the 75% of the smallest number of sequences for samples), and the tree nodes that prove more consistent across jackknifed datasets were deemed more robust.

1.1 Criteria for Patients/Controls' Pairs Selection.

An operational database, including microbiota OTUs distribution data from 96 faecal samples, 79 from controls e 17 from 6 patients' samples (3 samples collected for each patient, except in one case) was built up accordingly to age stratification groups (2-3; 4-6; 7-8; 9-10; 11-12; 13-16 years of age) and for each L2 (phylum)-L5 (Family)-L6 (species) taxonomic levels (Table 1).

TABLE 1
Correlation dataset between patient and control groups

| N | #SampleID | Age | Gender | Group | patient |
|---|---|---|---|---|---|
| 1 | N.11.9 | 2 | | 2_3 | Ver |
| 2 | N.11.1 | 2 | m | 2_3 | |
| 3 | N.11.2 | 2 | m | 2_3 | |
| 4 | N.11.3 | 2 | m | 2_3 | |
| 5 | N.11.4 | 2 | f | 2_3 | |
| 6 | N.11.8 | 2 | m | 2_3 | |
| 7 | N11.5 | 2 | f | 2_3 | |
| 8 | N11.6 | 2 | m | 2_3 | |
| 9 | N11.7 | 2 | f | 2_3 | |
| 10 | N.10.1 | 3 | m | 2_3 | |
| 11 | N.10.2 | 3 | f | 2_3 | |
| 12 | N.10.4 | 3 | m | 2_3 | |
| 13 | N.10.5 | 3 | f | 2_3 | |
| 14 | N.10.6 | 3 | m | 2_3 | |
| 15 | N10.3 | 3 | m | 2_3 | |
| 16 | N09.6 | 4 | m | 4_6 | |
| 17 | N09.7 | 4 | f | 4_6 | |
| 18 | N09.9 | 4 | f | 4_6 | |
| 19 | N.09.4 | 4 | m | 4_6 | |
| 20 | N08.1 | 5 | m | 4_6 | |
| 21 | N08.5 | 5 | f | 4_6 | |
| 22 | N07.5 | 6 | f | 4_6 | |
| 23 | N.07.3 | 6 | f | 4_6 | |
| 24 | N.07.4 | 6 | m | 4_6 | |
| 25 | N.07.6 | 6 | f | 4_6 | |
| 26 | N.06.1 | 7 | f | 7_8 | Deg |
| 27 | N.06.2 | 7 | m | 7_8 | |
| 28 | N.06.4 | 7 | m | 7_8 | |
| 29 | N.06.5 | 7 | m | 7_8 | |
| 30 | N.06.7 | 7 | m | 7_8 | |
| 31 | N.06.8 | 7 | f | 7_8 | |
| 32 | N06.6 | 7 | m | 7_8 | |
| 33 | N.05.1 | 8 | m | 7_8 | |
| 34 | N.05.2 | 8 | f | 7_8 | |
| 35 | N.05.3 | 8 | m | 7_8 | |
| 36 | N.05.4 | 8 | m | 7_8 | |
| 37 | N.05.5 | 8 | m | 7_8 | |
| 38 | N.05.6 | 8 | m | 7_8 | |
| 39 | N.05.7 | 8 | m | 7_8 | |
| 40 | N.05.8 | 8 | m | 7_8 | |
| 41 | N.05.9 | 8 | f | 7_8 | |
| 42 | N.04.1 | 9 | m | 9_10 | Pasc |
| 43 | N.04.2 | 9 | f | 9_10 | |
| 44 | N.04.3 | 9 | f | 9_10 | |
| 45 | N.04.4 | 9 | m | 9_10 | |
| 46 | N.04.5 | 9 | m | 9_10 | |
| 47 | N.04.6 | 9 | f | 9_10 | |
| 48 | N.04.7 | 9 | f | 9_10 | |
| 49 | N.04.8 | 9 | m | 9_10 | |
| 50 | N.03.03 | 10 | m | 9_10 | |
| 51 | N.03.1 | 10 | f | 9_10 | |
| 52 | N.03.2 | 10 | m | 9_10 | |
| 53 | N.03.4 | 10 | m | 9_10 | |
| 54 | N.03.5 | 10 | f | 9_10 | |
| 55 | N.03.6 | 10 | f | 9_10 | |
| 56 | N.03.7 | 10 | f | 9_10 | |
| 57 | N.03.8 | 10 | m | 9_10 | |
| 58 | N.02.1 | 11 | f | 11_12 | Cag |
| 59 | N.02.2 | 11 | f | 11_12 | Per |
| 60 | N.02.3 | 11 | f | 11_12 | Spar |
| 61 | N.02.4 | 11 | f | 11_12 | |
| 62 | N.02.5 | 11 | f | 11_12 | |
| 63 | N.02.6 | 11 | m | 11_12 | |
| 64 | N.02.7 | 11 | m | 11_12 | |
| 65 | N.02.8 | 11 | f | 11_12 | |
| 66 | N.01.1 | 12 | m | 11_12 | |
| 67 | N.01.2 | 12 | f | 11_12 | |
| 68 | N.00.1 | 13 | m | 13_16 | |
| 69 | N.00.2 | 13 | f | 13_16 | |
| 70 | N.00.3 | 13 | m | 13_16 | |
| 71 | N.00.4 | 13 | f | 13_16 | |
| 72 | N.00.5 | 13 | f | 13_16 | |
| 73 | N.00.6 | 13 | f | 13_16 | |
| 74 | N.98.3 | 14 | f | 13_16 | |
| 75 | N.99.1 | 14 | m | 13_16 | |
| 76 | N.99.2 | 14 | f | 13_16 | |
| 77 | N.98.1 | 15 | m | 13_16 | |
| 78 | N.98.2 | 15 | f | 13_16 | |
| 79 | N.97.01 | 16 | f | 13_16 | |

2 Question No 1: Can the Controls be Divided into Groups?

2.1 Statistical Methods.

A hierarchical cluster analysis with Wald's method has been performed in order to group controls into a limited number of homogeneous clusters. The cluster is characterized by multivariate data revealing characteristics of any structure or patterns present (e.g., microbiota profiles generating subgroups belonging to the same clustering tree node) (see Everitt, B. 2011. Cluster analysis. Chichester, West Sussex, U.K: Wiley. ISBN 9780470749913. Agresti, A. 2007. An Introduction to Categorical Data Analysis, 2nd ed., New York: John Wiley & Sons. The number of clusters was chosen by dendrogram computation. The null hypothesis of independence between gender and clusters and age groups (2-3, 4-6, 7-8, 9-10, 11-12, 13-16) and cluster was tested by chi square independence test. P-values were computed both analytically and by re-sampling (see Agresti, 2007).

Controls are clustered using L2, L5 and L6 taxon levels.

2.2 Main Results.

Clusters were independent on gender and dependent on age groups for all taxon levels. Therefore, at each taxon level it was meaningful to group controls by age and not by gender.

a) Clustering at L2 Taxon Level.

Main results: clustering was independent on gender and dependent on age group. So it is meaningful to group controls by age and not by gender.

FIG. 1.—Clustering of controls by Wald's method at L2 taxon level—3 groups (curly brackets from I to III) 6 groups (curly brackets from A to F).

Both with 3 and 6 clusters gender and groups resulted statistically independent as p-values were larger than 5%. Both with 3 and 6 clusters age and groups resulted statistically dependent as p-values were smaller than 1% (see Table 2).

TABLE 2
Chi square test of independence between clusters and age and
cluster and gender at L2 taxon level.

| | Chi | p. value | p. value |
|---|---|---|---|
| Categories | sq. Test | (exact) | (approx) |
| Age_groups vs. 3 clusters | 139.4 | <2.2 * 10\hat{ }⁻¹⁶ | <2.2 * 10\hat{ }−16 |
| Age_groups vs. 6 clusters | 277.24 | <2.2 * 10\hat{ }⁻¹⁶ | <2.2 * 10\hat{ }−16 |
| Gender vs. 3 clusters | 7.37 | 0.1173 | 0.0821 |
| Gender vs. 6 clusters | 10.91 | 0.3645 | 0.3585 |

p-value approximation is computed by 9999 resamplings

b) Clustering at L5 Taxon Level.

Main Results:

clustering was independent on gender and dependent on age group. Therefore, it was meaningful to group controls by age and not by gender.

FIG. 2.—Clustering of controls by Wald's method at L5 taxon level—3 groups (curly brackets from I to III) 6 groups (curly brackets from A to F).

Both with 3 and 6 clusters gender and groups resulted statistically independent as p-values were larger than 5%. Both with 3 and 6 clusters age and groups resulted statistically dependent as p-values were smaller than 1% (see Table 3).

TABLE 3
Chi square test of independence between clusters and age and
cluster and gender at L5 taxon level

| | Chi | p. value | p. value |
|---|---|---|---|
| Categories | sq. Test | (exact) | (approx) |
| Age_groups vs. 3 clusters | 144.85 | <2.2 * 10\hat{ }⁻¹⁶ | <2.2 * 10\hat{ }⁻¹⁶ |
| Age_groups vs. 6 clusters | 353.92 | <2.2 * 10\hat{ }⁻¹⁶ | <2.2 * 10\hat{ }⁻¹⁶ |
| Gender vs. 3 clusters | 4.97 | 0.2908 | 0.2983 |
| Gender vs. 6 clusters | 10.16 | 0.4265 | 0.4187 |

p-value approximation is computed by 9999 resamplings

c) Clustering at L6 Taxon Level.

Main results: clustering was independent on gender and dependent on age group. Therefore, it was meaningful to group controls by age and not by gender.

FIG. 3—Clustering of controls by Wald's method at L6 taxon level—3 groups (curly brackets from I to III) 6 groups (curly brackets from A to F).

Both with 3 and 6 clusters gender and groups resulted statistically independent as p-values were larger than 5%. Both with 3 and 6 clusters age and groups resulted statistically dependent as p-values were smaller than 1% (see Table 4).

TABLE 4
Chi square test of independence between clusters and age and
cluster and gender at L6 taxon level

| | Chi | | p. value |
|---|---|---|---|
| Categories | sq. Test | p. value (exact) | (approx) |
| Age_groups vs. 3 clusters | 120.38 | <2.2 * 10\hat{ }⁻¹⁶ | <2.2 * 10\hat{ }⁻¹⁶ |
| Age_groups vs. 6 clusters | 244.32 | <2.2 * 10\hat{ }⁻¹⁶ | <2.2 * 10\hat{ }⁻¹⁶ |
| Gender vs. 3 clusters | 6.99 | 0.14 | 0.08 |
| Gender vs. 6 clusters | 9.11 | 0.52 | 0.51 |

p-value approximation is computed by 9999 resamplings

3. Question No 2: Do the Samples from Each Patients Change Over Time?

Statistical Methods.

Three samples were collected from each patients at three different times (three consecutive days). By using the Kruskal-Wallis rank sum test (see Kruskal and Wallis, 1952) the null hypothesis that the median of the three samples is the same against the alternative hypothesis that they differed in at least one sample has been tested.

The test was performed on each patient and at L2, L5 and L6 taxon levels.

Main Result:

the medians of samples from all patients were the same at any time, i.e. they did not change over time at all taxon levels. All p-values were greatly higher than 10% (see Table 5).

TABLE 5
Kruskal - Wallis test on all patients at L2, L5, L6 taxon levels

| | | Kruskal Wallis chi | | |
|---|---|---|---|---|
| Patient | Taxon | sqare | df¹ | p-value |
| n^{o} 1 - Cag | L2 | 0.0099 | 2 | 0.9951 |
| | L5 | 0.9621 | 2 | 0.6181 |
| | L6 | 22.025 | 2 | 0.3325 |
| n^{o}2 - Deg | L2 | 0.0016 | 1 | 0.9678 |
| | L5 | 0.025 | 1 | 0.8743 |
| | L6 | 0.0347 | 1 | 0.8521 |
| n^{o}3 - Pas | L2 | 0.3553 | 2 | 0.8372 |
| | L5 | 0.6616 | 2 | 0.7183 |
| | L6 | 0.8649 | 2 | 0.6489 |
| n^{o}4 - Per | L2 | 0.0744 | 2 | 0.9635 |
| | L5 | 0.4667 | 2 | 0.7919 |
| | L6 | 13.238 | 2 | 0.5159 |
| n^{o}5 - Spar | L2 | 0.0829 | 2 | 0.9594 |
| | L5 | 0.0463 | 2 | 0.9791 |
| | L6 | 0.2370 | 2 | 0.8882 |
| n^{o}6 - Ver | L2 | 0.5957 | 2 | 0.7424 |
| | L5 | 0.4141 | 2 | 0.8130 |
| | L6 | 0.0282 | 2 | 0.9860 |

¹Degree of freedom

4. Question No 3: Comparison of OTUs Distributions Between Each Patient and Controls within the Same Age Group

Statistical Methods.

We compared the average of each patient's samples (OTUs distribution) and the average of samples of controls from the same age group. By Kruskal-Wallis rank sum test (see Kruskal and Wallis, 1952) we tested the null hypothesis that the medians of the two samples are the same against the alternative hypothesis that they differ at L2, L5 and L6 taxon levels.

Main Results:

as shown in Table 6, the difference between cases and controls was not statistically significant at L2 taxon level (p-values are larger than 10% in all patients). Such difference was statistically significant at L5 and L6 taxon level (p-values are smaller than 1% in all patients).

TABLE 6
Kruskal - Wallis test on each patient vs. controls in the same
age group at L2, L5, L6 taxon levels

| | | Kruskal Wallis | |
|---|---|---|---|
| Patient | Taxon | chi sqare | p-value |
| n^{o} 1 - Cag | L2 | 1.93 | 0.16 |
| | L5 | 19.62 | 9.43 * 10\hat{ }⁻⁶ |
| | L6 | 22.74 | 1.84 * 10\hat{ }⁻⁶ |
| n^{o}2 - Deg | L2 | 2.56 | 0.11 |
| | L5 | 24.01 | 9.56 * 10\hat{ }⁻⁷ |
| | L6 | 35.04 | 3.23 * 10\hat{ }⁻⁹ |
| n^{o}3 - Pas | L2 | 2.11 | 0.14 |
| | L5 | 2.08 | 5.51 * 10\hat{ }⁻⁷ |
| | L6 | 40.21 | 2.28 * 10\hat{ }⁻⁷ |
| n^{o}4 - Per | L2 | 0.70 | 0.40 |
| | L5 | 6.12 | 0.01 |
| | L6 | 9.52 | 2 * 10\hat{ }⁻³ |
| n^{o}5 - Spar | L2 | 0.83 | 0.36 |
| | L5 | 6.25 | 0.01 |
| | L6 | 3.27 | 0.07 |
| n^{o}6 - Ver | L2 | 1.21 | 0.27 |
| | L5 | 22.48 | 2.13 * 10\hat{ }⁻⁶ |
| | L6 | 26.04 | 3.34 * 10\hat{ }⁻⁷ |

5. Question No 4: Dysbiosis: Dissimilarity Measure Between Cases and Controls

Statistical Methods

As in Leti (1983), we used the percentage quadratic dissimilarity index

Z=(½*Σ(f_{case}−f_{controls})̂2)^{̂1/2}

where f_{case} is the OTUs distribution in a patient and f_{controls} is the OTUs distribution among controls in the same age group. This index varied between 0 and 1 and can be expressed in percentage. The value 0 means no dissimilarity and the value 1 means max dissimilarity. Therefore, this index is suitable to be used as a measure of dysbiosis.

We computed it only at L5 and L6 taxon levels and not at L2, because in previous section it has been proved that OTUs distributions are statistically different between each case and controls within the same age group at L5 and L6 taxon levels and not at L2 level.

Main Results

1. Patient Cag showed a dissimilarity degree versus controls at 35% (L6)-36% (L5) of maximum dissimilarity.

2. Patient Deg showed a dissimilarity degree versus controls at 38% (L6)-40% (L5) of maximum dissimilarity.

3. Patient Pas showed a dissimilarity degree versus controls at 26% (L5)-29% (L6) of maximum dissimilarity.

4. Patient Per showed a dissimilarity degree versus controls at 30% (L5)-31% (L6) of maximum dissimilarity.

5. Patient Spar showed a dissimilarity degree versus controls at 10% (L5)-28% (L6) of maximum dissimilarity.

6. Patient Ver showed a dissimilarity degree versus controls at 29% (L5)-36% (L6) of maximum dissimilarity.

TABLE 7
Dysbiosis or dissimilarity index between OTUs distribution in
each patient vs. OTUs distribution in controls in the same age
group at L5 and L6 taxon levels

| Patient | Taxon level | Dysbiosis index |
|---|---|---|
| n^{o} 1 - Cag | L5 | 0.3661 |
| | L6 | 0.3573 |
| n^{o}2 - Deg | L5 | 0.4001 |
| | L6 | 0.3842 |
| n^{o}3 - Pas | L5 | 0.2698 |
| | L6 | 0.2928 |
| n^{o}4 - Per | L5 | 0.3019 |
| | L6 | 0.3184 |
| n^{o}5 - Spar | L5 | 0.1074 |
| | L6 | 0.2795 |
| n^{o}6 - Ver | L5 | 0.2911 |
| | L6 | 0.3601 |

### EXAMPLE 2: EXTENSION OF MICROBIOTA PROFILING FROM CHILDHOOD TO ADULTHOOD

The method of comparing the patient microbiota profile to the healthy reference groups (CTRLs) was extended from the childhood age to the adulthood. With this aim, besides the groups of 2-3; 4-6; 7-8; 9-10; 11-12; 13-16 years of age, a group of controls from 17-70 years was added to the CTRLs groups, consistently with what recently described (N Engl J Med 375; 24, Dec. 15, 2016) and even improved in the range 12-16. Also in this group median values of OTUs distribution were calculated for each L2 (phylum)-L5 (Family)-L6 (species) taxonomic levels (data not shown). Accordingly, the dissimilarity percentage was calculated for the adult range in a way to apply the dysbiosis computation also to faecal samples collected by adult patients.

### REFERENCES

1. Putignani L, Del Chierico F, Vernocchi P, Cicala M, Cucchiara S, Dallapiccola B; Dysbiotrack Study Group. Gut Microbiota Dysbiosis as Risk and Premorbid Factors of IBD and IBS Along the Childhood-Adulthood Transition. Inflamm Bowel Dis. 2016 February; 22(2):487-504.
2. Sekirov I, Russell S L, Antunes L C, Finlay B B. Gut microbiota in health and disease. Physiol Rev. 2010; 90:859-904;
3. Dethlefsen L, Eckburg P B, Bik E M, Reiman D A. Assembly of the human intestinal microbiota. Trends Ecol Evol. 2006; 21:517-523
4. Blaut M, Collins M D, Welling G W, Dore J, Van L J, de Vos W. Molecular biological methods for studying the gut microbiota: The EU human gut flora project. Br J Nutr. 2002; 87(Suppl 2):S203-211;
5. McCartney A L. Application of molecular biological methods for studying probiotics and the gut flora. Br J Nutr. 2002; 88(Suppl 1):S29-S37
6. Peter H, Sommaruga R. An evaluation of methods to study the gut bacterial community composition of freshwater zooplankton. J Plankton Res. 2008; 30:997-1006
7. Arumugam M., Raes J., Pelletier E., et al. Enterotypes of the human gut microbiome. Nature. 2011; 473(7346):174-180. doi: 10.1038/nature09944
8. Siezen R. J., Kleerebezem M. The human gut microbiome: are we our enterotypes? Microbial Biotechnology. 2011, 4(5):550-553. doi: 10.1111/j.1751-7915.2011.00290.x.
9. Del Chierico F, Vernocchi P, Petrucca A, Paci P, Fuentes S, Pratico G, Capuani G, Masotti A, Reddel S, Russo A, Vallone C, Salvatori G, Buffone E, Signore F, Rigon G, Dotta A, Miccheli A, de Vos W M, Dallapiccola B, Putignani L). Phylogenetic and Metabolic Tracking of Gut Microbiota during Perinatal Development. PLoS One. 2015 Sep. 2; 10(9):e0137347. doi: 10.1371/journal.pone.0137347. eCollection 2015.
10. Woodmansey E. J. Intestinal bacteria and ageing. Journal of Applied Microbiology. 2007; 102(5):1178-1186. doi: 10.1111/j.1365-2672.2007.03400.x
11. Ercolini D, De Filippis F, La Storia A, Iacono M. “Remake” by high-throughput sequencing of the microbiota involved in the production of water buffalo mozzarella cheese. Appl. Environ. Microbiol. 2012; 78:8142-8145.
12. Caporaso, J G, Kuczynski, J, Stombaugh, J, Bittinger, K, Bushman, F D, Costello, E K, et al. QIIME allows analysis of high-throughput community sequencing data. Nat. Methods 2010; 7, 335-336.
13. Reeder J, Knight R. Rapidly denoising pyrosequencing amplicon reads by exploiting rank-abundance distributions. Nat. Methods. 2010; 7:668-669.
14. Edgar R C. Search and clustering orders of magnitude faster than BLAST. Bioinforma. Oxf. Engl. 2010; 26:2460-2461.
15. Agresti, A. 2007. An Introduction to Categorical Data Analysis, 2nd ed., New York: John Wiley & Sons.
16. Everitt, B. 2011. Cluster analysis. Chichester, West Sussex, U.K: Wiley. ISBN 9780470749913.
17. Kruskal, W H and Wallis, A. 1952. “Use of ranks in one-criterion variance analysis”. Journal of the American Statistical Association. 47: 583-621.
18. Leti, G. (1983), Elementi di statistica descrittiva, II Mulino, Milano.

1) Method for providing a gut microbiota reference control tool of healthy subjects for in vitro diagnosis of gut dysbiosis index or percentage, said method comprising or consisting of:
a) clustering gut biological, samples of healthy subjects in one or more clusters wherein, when the age, of the healthy subjects is less than 17 or 17±2 years, preferably from 18 months to less than 17 or 17±2 years, the gut biological samples belong to healthy subjects having an difference less than 4 years, preferably less than 3 years, more preferably less than 2 years, among them in each cluster, and/or in a further cluster wherein the gut biological samples belong to healthy subjects whose age ranges from 17, or 17±2 years, to 70 or 70±2 years;
b) detecting by metagenomics the identity and frequency of all phyla, families and species of gut microbiota in the gut biological samples of each of said healthy subjects of each of said one or more clusters; and
c) calculating the median values of the operational taxonomic units distribution for each of said one or more clusters and/or said further cluster.

2) Method according to claim 1, wherein said one or more clusters are clusters wherein the gut biological samples belong to healthy subjects whose age ranges from 2 years to less than 4 years, from 4 years to less than 7 years, from 7 years to less than 9 years, from 9 years to less than 11 years, from 11 years to less than 13 years, from 13 years to less than 17 years, and/or from 17 years to 70 years.

3) Method according to claim 1, wherein the gut biological samples are chosen from the group consisting of faecal samples, gut tissue samples, preferably faecal samples.

4) Method according to claim 1, wherein the healthy, subjects come from the same Nation.

5) Gut microbiota reference control tool of healthy subjects for in vitro diagnosis of gut dysbiosis index or percentage, said reference control tool comprising or consisting of the median values of the operational taxonomic units distribution of all phyla, families and species, which are detected by metagenomics of gut microbiota in gut biological samples of healthy subjects, wherein said gut biological samples are clustered in one or more clusters wherein, when the age of the healthy subjects is less than 17 or 17±2 years, preferably from 18 months to less than 17 or 17±2 years, the gut biological samples belong to healthy subjects having an age difference less than 4 years, preferably less than 3 years, more preferably less than 2 years, among them in each cluster, and/or in a further cluster wherein the gut biological samples belong to healthy subjects whose age ranges from 17, or 17±2 years, to 70 or 70±2 years; wherein said median values of the operational taxonomic units distribution are the median values of the operational taxonomic units distribution for each of said one or more clusters and/or said further cluster.

6) Gut microbiota reference control tool according to claim 5, wherein said one or more clusters are clusters wherein the gut biological samples belong to healthy subjects whose age ranges from 2 years to less than 4 years, from 4 years to less than 7 years, from 7 years to less than 9 years, from 9 years to less than 11 years, from 11 years to less than 13 years, from 13 years to less than 17 years, and/or from 17 years to 70 years.

7) Gut microbiota reference control tool according to claim 5, wherein the gut biological samples are chosen from the group consisting of faecal samples, gut tissue samples, preferably faecal samples.

8) Gut microbiota reference control tool according to claim 5, wherein the healthy subjects come from the same Nation.

9) Method for in vitro diagnosis of gut dysbiosis index or percentage comprising or consisting of:
a) detecting by metagenomics the identity and frequency of all detectable phyla, and species of gut microbiota in 3 gut biological samples of a patient which are collected in consecutive days;
b) calculating the median values, of operational taxonomic units distribution of said all detectable phyla, families and species of said gut biological samples of the patient;
c) calculating the dissimilarity index or percentage of the median values of the operational taxonomic units distributions of gut microbiota of the patient in comparison with the median values of the operational taxonomic units distribution of a cluster of the gut microbiota reference control tool of healthy subjects as defined in claim 5, wherein said cluster is that in which the age of the patient falls in the age range of the healthy subjects of the same cluster.

10) Method according to claim 9, wherein the dissimilarity index or percentage is calculated for said all phyla, families and species of gut microbiota of the patient by the formula:
Z=(½×Σ(f_{case}−f_{controls})²)^{1/2}
or
Z=(½×Σ(f_{case}−f_{controls})²)^{1/2}×100
wherein f_{case} is the median value of the operational taxonomic units distribution of said all phyla, families and species of gut microbiota of the patient;
and f_{controls} is the median value of the operational taxonomic units distribution of all phyla, families and species of gut microbiota of the cluster of the gut microbiota reference control tool of the healthy subjects, wherein said cluster is which the age of the patient falls in the age range of the healthy subjects of the same cluster.

11) Method according to claim 9, wherein the patient comes from the same Nation of the healthy subjects of the control tool.
